(19) United States
(12) Patent Application Publication
(10) Pub. No.: US 2004/0122445 A1
(43) Pub. Date: Jun. 24, 2004

(54) Rigid extractor

(75) Inventors: Gary L. Butler, Bloomington, IN (US); Thomas L. Foster, Poland, IN (US); Frank J. Fischer JR., Bloomington, IN (US)

Correspondence Address: GENERAL NUMBER 00757, BRINKS HOFER GILSON & LIONE, P.O. BOX 10395, CHICAGO, IL, 60611, US

(73) Assignees: Vance Products, Inc.; Cook Urological, Inc.

(21) Appl. No.: 10/679,007

(22) Filed: Oct. 3, 2003

Related U.S. Application Data
(60) Provisional application No. 60/416,035, filed on Oct. 4, 2002.

(51) Int. Cl. A61B 17/22

(52) U.S. Cl. 606/127

(57) ABSTRACT

A rigid extractor is revealed, a rigid device for use in percutaneous procedures to remove kidney stones directly from the kidneys. The rigid extractor uses an outer rigid cannula and an inner cannula to control a basket retriever for removing kidney stones and calculi from a kidney of a patient. The extractor is desirably used with a fluoroscope, in which the surgeon maneuvers the extractor with the aid of a view of the operating field provided by the fluoroscope. The surgeon then maneuvers the extractor to grasp the kidney stones and remove from the patient. The extractor may also be used with a nephroscope. The device may also be used with a retrieval assembly other than a basket, such as a pair of jaws, or a pair of scissors.

This application claims the benefit of the filing date under 35 U.S.C. § 119(e) of Provisional Application No. 60/416,035, filed Oct. 4, 2002, entitled Rigid Extractor, which is hereby incorporated by reference.

### FIELD OF THE INVENTION

This invention relates generally to surgical retrieval devices. The device relates more particularly to devices for capturing and retrieving or extracting stones, calculi, concretions, foreign bodies and the like from a human or veterinary patient. The device may also be useful for biopsies and other surgical retrievals.

### BACKGROUND OF THE INVENTION

Various organs and passages in the body are subject to the development of stones, calculi and the like. For example, kidney stones are a common problem in the United States. Kidney stones are painful and are the most frequent cause of kidney inflammation. Calculi and concretions in other parts of the biliary system are also commonplace. Similarly, stones, calculi, concretions and the like can develop throughout the renal or urinary system, not only in the ureters and distal to them, but also in the renal tubules and in the major and minor renal calyxes.

Minimally invasive surgical procedures have been developed for the removal of stones, calculi, concretions and the like from the biliary, vascular, and urinary systems, as well as for the removal or retrieval of foreign bodies from a variety of locations in the body. Such procedures avoid the performance of open surgical procedures such as, for example, an anatrophic nephrolithotomy. Minimally invasive procedures can instead employ percutaneous access, in which stones, calculi, concretions, foreign bodies and the like are removed through a percutaneously inserted access sheath. Several access routes are suitable, depending upon the specific system and the particular location in the system at which the stones, calculi, concretions, foreign bodies or the like are found. One access route that is infrequently used is direct percutaneous insertion of a retrieval device to remove calculi and kidney stones.

Without regard to the particular access route, percutaneous extraction may be based upon the use of catheters or similar devices to engage and remove the stones, calculi, concretions, foreign bodies and the like. Such catheters and devices typically comprise a hollow, flexible sheath and a plurality of wires positioned in and extendable from the sheath. The wires are joined or arranged so as to form a means, such as a basket or forceps for engaging the object to be retrieved when the wires are extended from the sheath. The wires may also form a continuum with the sheath. The engagement means (for example, a basket) can be collapsed by withdrawing the wires into the sheath. A helical basket permits entry of the stone or the like from the side of the basket, while an open ended (“eggwhip”) basket allows a head-on approach to the stone or the like. Other retrievers and graspers can include forceps or can include a loop or snare for encircling the body to be removed, the loop or snare being made of the wire. Such devices may be used in conjunction with a nephroscope, to aid the physician in seeing the operating field. Using such a device also tends to limit the size of the cannula and basket used.

Despite their successful use for some time, such retrieval devices are subject to drawbacks. The principal device that is used to retrieve kidney stones is a 3-pronged grasper. The prongs of the grasper, useful in grasping stones, may cause damage to kidney or contiguous tissue, leading to bleeding, and potentially significantly extending the time for the procedure. The very flexible, movable nature of these graspers adds to the problem, in that their flexibility and mobility make them more difficult to control.

It would be highly desirable to have a more controllable device for use inside the human body for the capture and retrieval or extraction of kidney stones and related calculi. The device preferably would not have sharp points that could scratch or puncture bodily tissue, and would be able to remove kidney stones up to one-quarter inch in diameter or even larger.

### BRIEF SUMMARY OF THE INVENTION

The foregoing problems are solved and a technical advance is achieved in a rigid extractor useful for capturing and extracting, retrieving or removing objects such as stones and calculi from the human body, and from kidneys in particular. Of course, the device is not limited to human bodies, but may also be used in veterinary applications. One embodiment is an extractor for removing an object from a location within a body. The extractor comprises an inner cannula, and at least one superelastic wire leg attached to the inner cannula. The extractor also comprises at least one superelastic wire loop attached to the inner cannula, and means for restricting relative movement of the at least one wire loop and the at least one wire leg, the movement restricting means being continuously and kinklessly formed with at least one of the at least one wire leg and the at least one wire loop, wherein the at least one superelastic wire leg, the at least one superelastic wire loop, and the means for restricting relative movement form a reversibly collapsible, tipless, atraumatic basket. The extractor also comprises a rigid outer cannula for delivering the basket to the location of the object.

Another embodiment of the invention is a rigid extractor for removing an object within a body. The extractor comprises an inner cannula, and a basket made of wires attached to the inner cannula. The extractor also comprises a rigid outer cannula for delivering the basket to the object, the rigid outer cannula containing the inner cannula, and a handle having a first end and a second end, the first end attached to the outer cannula and the second end attached to the inner cannula. Another embodiment comprises a method for removing an object from a body. The method comprises forming a passage with at least one of a needle, a wire guide, and an access sheath. The method then comprises inserting a rigid extractor near the object, and extending a basket from the rigid extractor and maneuvering the basket near the object. The method then comprises capturing the object with the basket, and removing the object from the body.

There are many ways to practice the present invention, as shown in the following drawings and specification. The embodiments described below are not meant to limit the invention, but rather to describe and illustrate the many ways that the present invention may be used. The advantages of the invention include better control over the retrieval device used at the distal end of the cannula, as well as better retrieval devices themselves, leading to easier entry, less damage and bleeding, and shorter removal procedures.

### BRIEF DESCRIPTION OF THE FIGURES

The present invention will now be described in conjunction with the following drawings, wherein like reference characters refer to like parts throughout the several views.

FIG. 1 is a plan view of a first embodiment of a rigid extractor.

FIG. 2 is a plan view of an inner cannula according to the embodiment of FIG. 1.

FIG. 3 is a perspective view of a portion of the handle according to FIG. 1.

FIGS. 4a and 4b are perspective and cross-sectional views of the rigid outer cannula according to FIG. 1.

FIGS. 5-10 are embodiments of loops for the wires forming a basket for the retrieval device of FIG. 1.

FIG. 11 is another embodiment of the rigid extractor.

FIG. 12 is another embodiment of the rigid extractor.

FIGS. 13 and 14 are grasper embodiments of the rigid extractor.

FIG. 15 is a graph of actuation force as a function of handle thickness.

FIG. 16 is a flowchart for a method of using the rigid extractor.

FIGS. 17-21 are alternate embodiments of a basket for the extractor.

### DETAILED DESCRIPTION OF THE PRESENTLY PREFERRED EMBODIMENTS

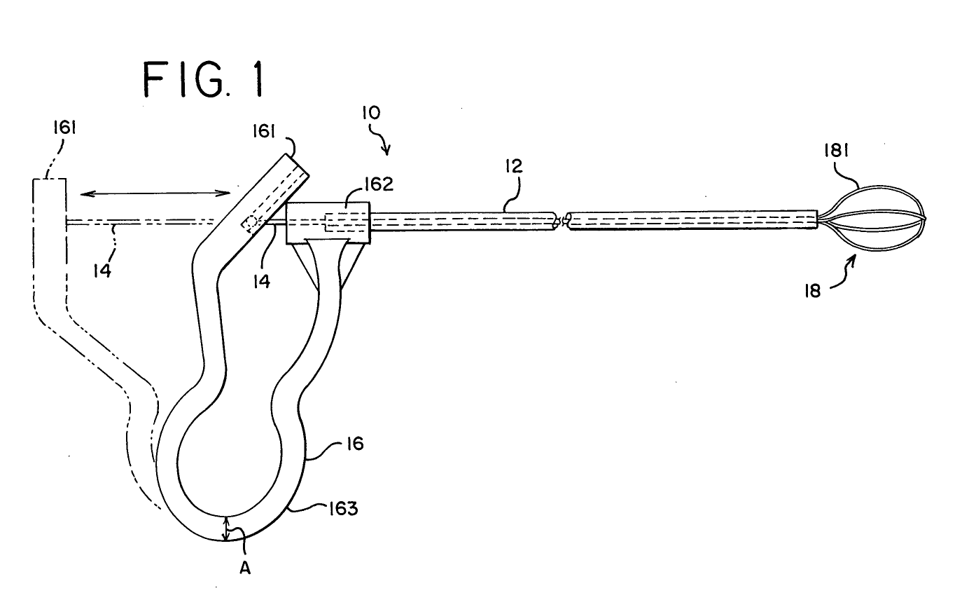

One embodiment of the invention is a rigid extractor useful in grasping and removing kidney stones from a patient. The kidney stones will typically have been reduced in size by a procedure in which laser energy, electrohydraulic energy, or sound energy is applied to reduce the stones in size for easier removal. A first embodiment is depicted in FIG. 1. The rigid extractor 10 comprises a rigid outer cannula 12 and an inner cannula 14. The inner cannula is attached to a basket 18 formed from at least one wire loop 181. The extractor also has a handle 16 for operating the extractor. The handle comprises a first end 161 attached to the inner cannula 14, and a second end 162, attached to the outer cannula 12. The handle also comprises a gripping portion with a flexible section 163. The basket is tipless, in the sense that there is no distal “end” to the basket in which the wires are secured to each other by soldering, welding, brazing, adhering, or the like.

The extractor is operated by applying hand pressure to the handle, squeezing the handle, deflecting the first end to the right in FIG. 1, and causing the inner cannula to translate to the right, and extending the basket. The handle is shown in solid line in the “squeezed” position, and in dotted line in the “relaxed” condition. It is understood that the basket is extended from the outer cannula as shown when the operator or surgeon applies pressure and squeezes the handle. When no pressure is applied to the handle, it is in a relaxed state, and the basket is collapsed within the outer cannula. The handle is not meant for insertion into the body of a patient, but remains outside the body during procedures for removing objects from a body. The handle preferably is made of nylon or other acceptable plastic. The handle shown in FIG. 1 has a length of about 3.5 inches (up and down) and the gripping portion has an inner diameter of about 1.05 inches and an outer diameter of about 1.50 inches. The thickness of the handle, in the direction of arrow A in FIG. 1, determines the force needed to deflect the handle and extend the basket from the cannula. In a preferred embodiment, the thickness of the handle is 0.225 inches, but it may also be from about 0.20 inches to about 0.25 inches, and may range from about 0.15 inches to about 0.30 inches. The width of the handle, perpendicular to the thickness direction shown, is preferably about 0.25 inches, but is not of particular importance, and may vary from about 0.125 inches to about 1 inch.

The thickness of the handle is important because the thickness determines the force required to deflect the handle and extend the basket. This force should be sufficient so that movement of the handle, and thus the basket, or other retrieval assembly on the distal end of the inner cannula, is deliberate but not difficult. In the course of conducting many tests, it was determined that a force of about five pounds is particularly preferred, while a force from about one pound to about eight pounds could be conveniently used. A force of about five pounds is necessary to deflect the handle if it is made from nylon 6, in a thickness of about 0.225 inches. When the handle is made with a thickness of about 0.150 inches, a force of about 1 pound is sufficient to operate the extractor. A thickness of over about 0.25 inches requires even greater force. The preferred thickness of the handle is therefore from about 0.20 to about 0.25 inches, preferably about 0.225 inches, and nylon 6 is a preferred material.

The outer cannula is a desirably rigid hollow tube that does not deflect appreciably in use. The extractor may be used with a nephroscope, in which the surgeon inserts the rigid extractor and its outer cannula into an appropriate channel in the nephroscope. The nephroscope allows the surgeon to view the operating field as the surgeon maneuvers the nephroscope and the extractor to capture and remove objects within the body, such as kidney stones. The outer cannula is sufficiently rigid for the surgeon to deflect and maneuver the nephroscope by using the outer cannula of the rigid extractor. The outer cannula is desirably made from a medically acceptable material such as stainless steel or stiff plastic material, preferably those with minimal coefficients of friction, such as reinforced plastic, stiff polyimide, PTFE, and other medically acceptable materials. 316 stainless steel is a preferred material. The outer cannula may vary in length from about 20 cm to about 60 cm. An intermediate length of about 38 cm works well with most patients and is preferred.

The outer cannula preferably has a wall thickness of at least 0.010 inches, desirably 0.014 inches, and more preferably 0.015 inches. The greater the wall thickness, the more rigid will be the outer cannula. This rigidity enables the surgeon to control the nephroscope and to maneuver the nephroscope into a desired position. The surgeon thus delivers the outer cannula and the basket to the desired location within the operating field. The outer cannula may preferably have an outer diameter from about 0.110 inches to about 0.200 inches, or from about 8.5 Fr to about 15.5 Fr. The inner diameter of the outer cannula depends on the dimension of the outer cannula and sufficient wall thickness to maintain the desired rigidity. Therefore, the inner diameter of the outer cannula may preferably range from about 0.080 inches to about 0.175 inches (from about 6 Fr. to about 13.5 Fr.). It is understood that wall thicknesses are preferably maintained at a minimum of 0.015 inches, but wall thicknesses slightly less than 0.015 inches may also be used.

The outer cannula may be covered with a thin adherent plastic covering, in order to aid the physician in placing the extractor. The covering is desirably a medical grade plastic material, such as Teflon® (PTFE) or other grade of plastic or fluoropolymer. These may include FEP, fluorinated ethylene propylene, PFA, perfluoroalkoxy polymer, and other medically-acceptable grades of thermoplastic or thermoset coatings. The covering is desirably thin, preferably about 0.10 to about 0.4 mm thick (about 0.004 to about 0.015 inches thick). FIGS. 4a and 4b depict an outer cannula 12 and a thin plastic covering 14 on the outer diameter of the cannula.

The inner cannula is preferably a solid rod, also made from stainless steel, although a hollow rod or tubing may also be used. The outer diameter of the inner cannula must fit within the inner diameter of the outer cannula, with sufficient clearance for easy movement within the outer cannula. The inner cannula is desirably at least about 0.065 inches in diameter (about 5 Fr). Other diameters may be used. The inner cannula may also have a short portion on its distal end hollowed out so that the wire loops and legs used to make the basket may fit into the distal end of the inner cannula. The wires are then desirably crimped to the inner cannula. They may also be secured to the inner cannula with an adhesive, such as a medically-acceptable grade of cyanoacrylate adhesive. Loctite 4011 works well and is preferred.

Of course, other embodiments of the rigid extractor may be smaller. One embodiment of a rigid extractor outer cannula has an outer diameter of about 4.5 Fr, about 0.059 inches, and an inner diameter of about 0.0465 inches with a nominal wall thickness of about 0.0065 inches. In this embodiment, the inner cannula had an outer diameter of about 0.0425 inches and an inner diameter of about 0.0315 inches. In order to make the cannula a little stiffer on the proximal end, a plug about 3 or 4 inches long was adhered to the proximal end of the inner cannula, near the point where it attaches to the handle. The particular embodiment was made of 316SS. Other materials suitable for the application may also be used. While this cannula is less rigid than one with walls 0.010 inches thick, it is much easier to control than a “flexible” cannula.

It is understood that the term leg may be used in two ways. The term leg may be used to describe a wire that is secured to the inner cannula and that extends from the distal end of the inner cannula to an area of the basket at which the leg is terminated and secured to another wire. The term leg may also be used to describe a portion of a wire that makes a complete loop from the inner cannula, to the center of the tipless basket, and then back to the inner cannula. Both ends of the wire in this case are secured to the inner cannula in the manner described above.

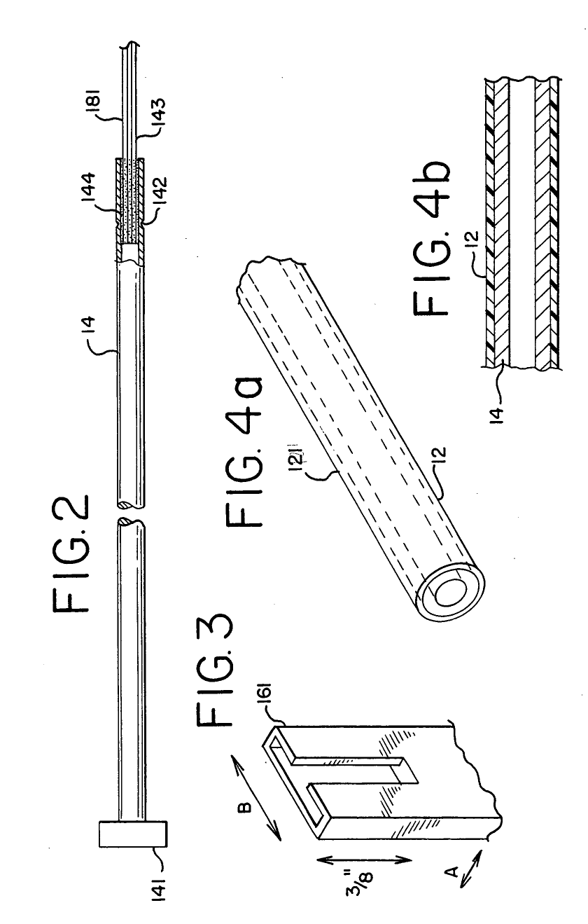

FIG. 2 depicts an inner cannula 14 with a T-shaped fitting 141 on the proximal end of the inner cannula, for fitting into a matching slot in the first end 161 of the handle 16. The inner cannula 14 also has at least one wire 181 secured to the cannula by a crimp 142 at the distal end of the cannula. The wires may also be secured with adhesive 144 as shown. FIG. 3 depicts a close view of the first end 161 of the handle used to operate the rigid extractor. The first end 161 includes a hollow portion forming a slot as shown, to receive the T-shaped fitting of the proximal end of the inner cannula. The first end may have a thickness designated in the direction of arrow A, and may have a width as shown in the direction of arrow B. The slot may extend a short distance into the first end of the handle, the distance being sufficient so that the inner cannula is not easily dislodged from the first end. A distance from about 0.25 inches to about 0.50 inches (about 6 to about 13 mm) is sufficient; about 0.375 inches (9-10 mm) is preferred.

The handle, the outer cannula, and the inner cannula cooperate to extend the basket from the outer cannula and retract the basket. The basket preferably is made so that it extends about 2.7 cm plus or minus 2 mm (about 1.05 inches plus or minus about 0.08 inches). Other extensions may be used. The basket will extend to the extent that the inner cannula is moved by the surgeon applying force and translating the inner cannula inside the outer cannula. Because the wires necessarily are not straight, but curve to form a basket, it is necessary for a translation of about 4 cm (about 1.6 inches) on the inner cannula to extend a basket of about 2.7 cm (about 1.1 inches). The handle should be designed and made so that squeezing the handle causes the first end 161 to deflect the desired amount by the time the first end contacts the second end 162 and no further translation of the inner cannula or the basket is possible. In a preferred embodiment, when the basket extends about 2.7 cm (about 1.1 inches) from the end of the outer cannula, the width of the basket (diameter) is about 1.8 cm, plus or minus about 2 mm (about 0.71 inches plus or minus about 0.08 inches). Other configurations may be used.

The wires used to form the basket are preferably a superelastic shape-memory material, such as Nitinol, a Ni—Ti alloy. Other alloys, such as Cu—Zn—Al, or Cu—Al—Ni may also be used. Round wires are preferably used to form the basket, but triangular and flat wires may also be used. Wires having a diameter of from about 0.08 mm to about 0.15 mm (about 0.003 inches to about 0.006 inches) are preferred, because their use permits a very small diameter basket, and hence a small diameter cannula. It is also preferred that the wires and the small loops used to restrict movement of the wires be kink-free. This is achieved by using the shape-memory metals mentioned above, and heat treating them in the desired shape for a short period of time.

Shape-memory or superelastic materials are heat treated or annealed from a weak (martinsite) structure to a strong (austenite) structure. The alloys are weak and deformable in the martinsitic state, which is thus useful for forming the basket and the loops. After transformation to the strong or martensitic state, they exhibit a superelastic property so long as the material remains above a transformation temperature, at which temperature it will revert to the martensitic state. The transformation temperature is desirably a low temperature, well below the temperature of a human body, and preferably below room temperature, about 20-25° C. The transformation temperature of the wires and the basket is thus selected to be below the operating temperature of the basket, thus keeping the basket in a superelastic state. In this state, the wires advantageously return to their original, unstressed shape when deforming stresses are removed. The superelastic wire alloy also increasingly resists deformation as the stress load is increased. Thus, when a superelastic basket is collapsed and placed into the cannula, a stress load is placed on the basket. When the basket is deployed, the stresses are removed, and the basket returns to the desired shape.

The baskets are formed by shaping the wires and loops into the desired shape at room temperature or below, preferably with one or more cold mandrels, and then annealing the properly-shaped basket at the proper annealing temperature for a time sufficient for the transformation to a superelastic state. In one example, a basket is formed from 0.15 mm diameter (about 0.006 inches) Ni—Ti Nitinol wire and is annealed at 800° F. (about 427° C.) for about 10 minutes. The time and temperature for annealing will vary with the alloy selected and with the diameter (thickness) of the wire. The basket itself, not the annealing oven, must remain at the desired annealing temperature for the proper length of time for annealing to be complete. Proper annealing is very important for the wires and the loops to remain kink-free during deployment and operation of the basket. If kinks form for any reason, it may be difficult to deploy (expand) or retract the basket.

The basket is desirably formed before the annealing operation, as discussed above, including all wires and loops. It is preferred for the small loops formed in the wires to be arranged so that the loops are on the inside of the basket, rather than the outside. Having small loops on the inside of the basket is advantageous in two ways. The loops are less likely to become kinked during basket deployment and maneuvers. And the basket and extractor are less likely to cause trauma to tissue that is contacted by the basket, i.e., the basket and the extractor are then atraumatic. Of course, the loops are not likely to cause trauma even if they are outside the basket, but they are preferred on the inside.

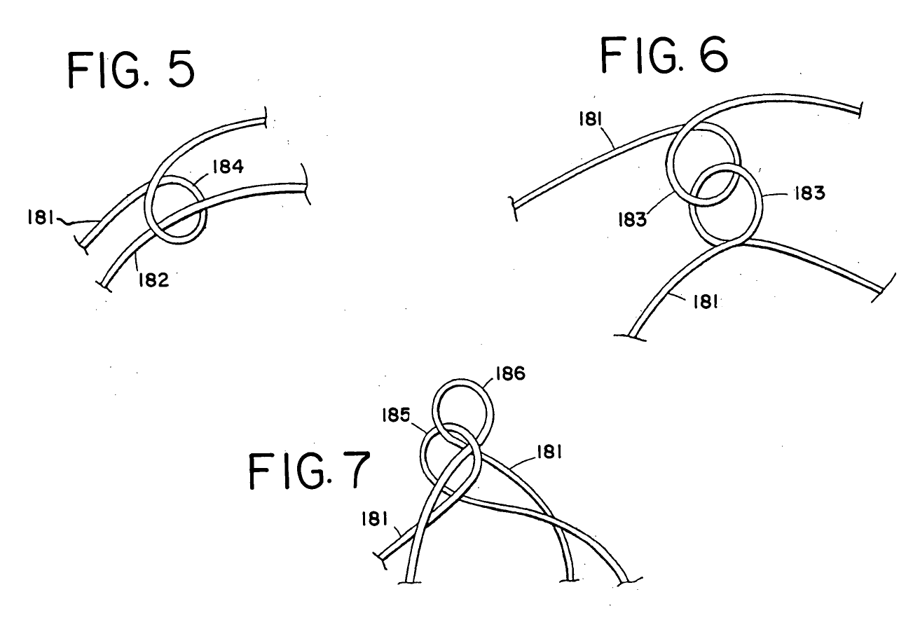

FIGS. 5-10 depict several embodiments of small loops that may be used to restrict movement of the wires, large loops, and legs that form the basket for the rigid extractor. In FIG. 5, a basket is formed from two large loops 181, 182, wherein large loop 181 is formed with an integral small loop 184 that encircles the other large loop 182. The diameter of the small loop is desirably formed as small as possible without kinking. FIG. 6 depicts a basket formed from two wires 181, each formed with a small loop 183 that encircles the other small loop. In both FIGS. 5 and 6, the small loops will coincide with the outer portion of the basket formed.

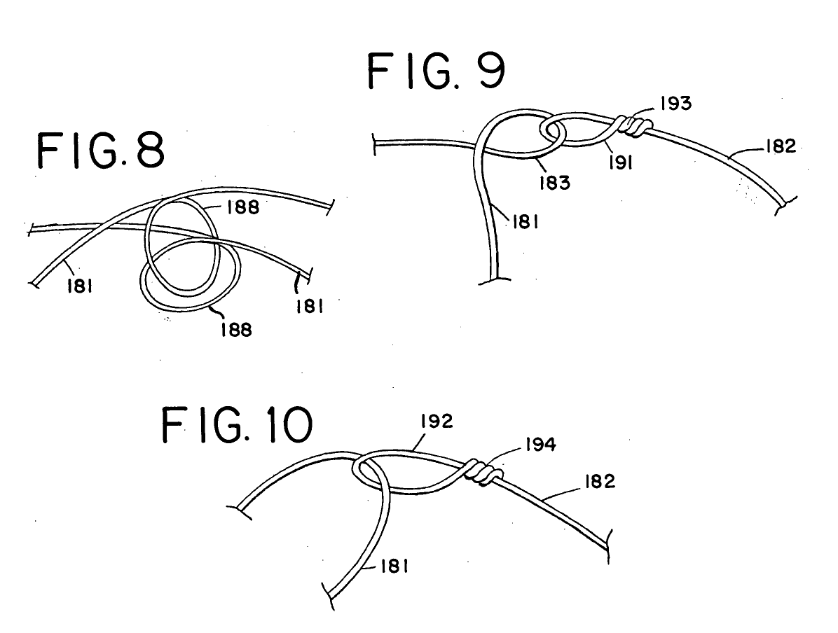

In FIG. 7, a first wire 181 is formed with a small loop 185 and a second wire 181 is formed with a small loop 186, the small loops intertwined with the wires in such a manner that the loops are external to the basket, that is, the small loops depend outwardly from at least one of the large loops. This is not a preferred embodiment, because the small loops desirably are formed inside the basket, and thus preferably depend inwardly from the large loops. Such a desirable configuration is depicted in FIG. 8. In this preferred embodiment, a first wire 181 is formed with a small loop 188 and a second wire 181 is also formed with a small loop 188. The small loops intertwine as shown, and will be contained within the basket, i.e., the small loops will depend inwardly from the large loops.

The embodiments of FIGS. 5-8 have used large-loop wires, in which a wire starts at the inner cannula with one end, forms part of a basket at its middle, and terminates at the inner cannula with the other end of the wire. Other embodiments of the basket may use a single “leg,” in which a wire starts at the inner cannula at one end, and then terminates at the basket, as shown in FIGS. 9-10. In FIG. 9, a large loop of wire 181 is formed with a small loop 183, while a wire leg 182 terminates with a small loop 191, the small loops 183, 191 intertwining and acting to restrict movement of both the wire loop 181 and the leg 182. In both FIG. 9 and FIG. 10, leg 182 should be terminated back upon itself in a joint 193, 194 that has no sharp edges or burrs. This will ensure that the basket and the extractor will remain atraumatic.

The rigid extractor with tipless, atraumatic, shape-memory basket may be used with a nephroscope, as mentioned above, or it may also be used directly, with a fluoroscope to aid the surgeon in manipulating the extractor to find, encircle, and remove a kidney stone or other object within a body. The method is preferably used after lithotripsy, in which the kidney stones are reduced in size by the application of sound energy, laser energy, electrohydraulic energy, or other outside source of energy to reduce the stones in size. In one method of using the extractor, a needle is inserted below the 12th rib of a patient. A wire guide is inserted into the region of interest, and the opening is dilated sequentially by a series of small but increasingly larger tubes. In this manner, a final tube up to 26 Fr to 30 Fr may be used. If desired, an access sheath, such as a PTFE access sheath, may be subsequently inserted and the dilation tube removed. The rigid extractor is then inserted through the access sheath. As mentioned above, nephroscopy and a nephroscope may be used instead of fluoroscopy.

As noted above, the rigid cannula is expected to find use in procedures for removing kidney stones from patients. The rigid extractor may also be used in other applications, such as the urinary, biliary, vascular or other systems. The details of the construction or composition of the various elements of the rigid extractor, the outer cannula, the inner cannula, and the basket, not otherwise disclosed are not believed to be important to the achievement of the advantages of the present invention, so long as the elements possess the strength or rigidity or elasticity, as described above, as needed to perform as desired. The selection of such details of construction are believed to be well within the ability of one having skill in the art, in view of the present disclosure.

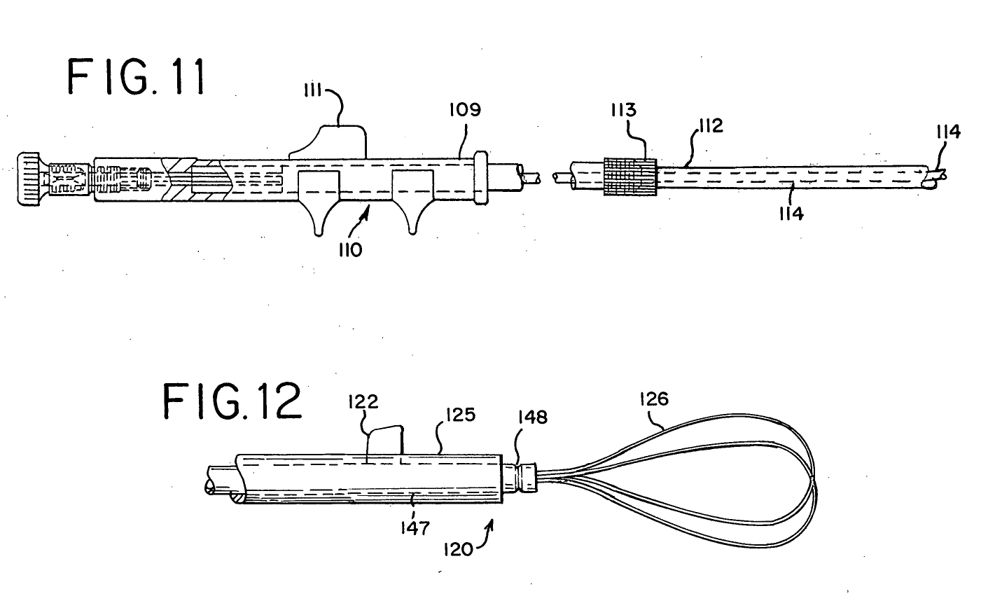

FIGS. 11 and 12 feature alternate embodiments of the rigid extractor. While the handle depicted in FIG. 1 is preferred, other handles and configurations may be used. FIG. 11 depicts an embodiment of a rigid extractor 110 in which the outer cannula 112 is fixed to a handle 109 with a fitting 113 while the proximal end of the movable inner cannula 114 (shown in dotted line) is attached to a control button 111. A tipless, atraumatic basket (not shown) is attached to the distal end of the inner cannula. FIG. 12 depicts an even simpler embodiment 120 of a tipless atraumatic basket 126 with a rigid outer cannula 125. In this embodiment, the basket 126 is affixed to the inner cannula 147 by a crimp joint 148. The inner cannula 147 and control button 122 are used to deploy the basket 126 from the outer cannula 125 to encircle and remove stones or calculi from a body.

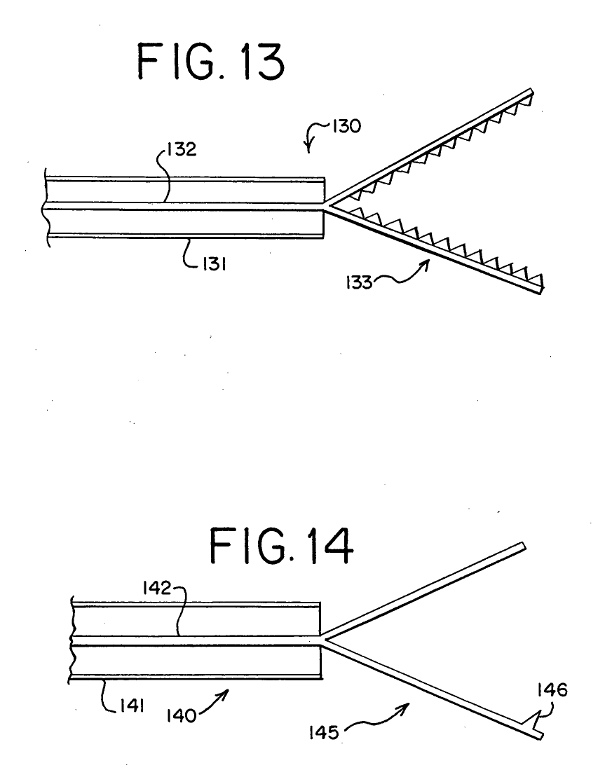

Other embodiments may also be made using retrieval devices other than a basket, such as a jaw-type retrieval assembly or a scissors-type retrieval assembly. A grasper assembly could also be used, and although a basket-type retrieval assembly may be preferable, a grasper that is easier to place and control by a surgeon may have its place among medical retrieval devices. A rigid cannula and controlled-force handle may be used with these other retrieval assemblies, as depicted in FIGS. 13 and 14. FIG. 13 depicts an extractor 130 with a jaw-type retrieval device 133 for grasping an object within a body. A user extends the jaws from the rigid outer cannula 131 and actuates the jaws using inner cannula 132 and control handle 161. Shape-memory metals may be used so that the jaws are in a relaxed state when extended from the outer cannula and in a stressed state when they are in the cannula. FIG. 14 is an embodiment of an extractor 140 with a scissors-type retrieval assembly 145 for cutting, as for a biopsy. In this embodiment, the scissors-type retrieval assembly is controlled by the inner cannula 142 and control handle 161. When the scissors are extended from the outer cannula 141, they separate and may be used for cutting. The scissors may be equipped with a “mouse tooth” 146 for impaling an object within a body. Shape-memory metals may be used so that the scissors are in a relaxed state when extended from the outer cannula 161 and in a stressed state when they are in the cannula. The basket described above, and the jaws and the scissors described here, may be considered as retrieval assemblies or devices at the distal end of the inner cannula. The rigid outer cannula is used to maneuver the extractor near the object to be removed, so that the retrieval assembly, whether basket, jaws, scissors, or grasper, or other retrieval device, may be used to remove the object.

It was noted above that the thickness of the handle (in FIG. 1) determines the force that the surgeon uses to extend the basket from the sheath. If the handle, the inner cannula, the outer cannula, and the basket are relatively free of friction, then the potential energy stored in the “squeezed” handle is available for grasping a stone or other calculus. This force used to squeeze the handle is stored as potential energy in the deformation of the handle, much as energy is stored in a compressed spring. That energy or force is applied to the stone or calculus when the surgeon releases the handle and the potential energy is used to trap or “squeeze” the stone or calculus, or to operate another retrieval assembly at the distal end of the inner cannula. The force desired is typically that force which is sufficient to trap and hold, but not sufficient to crush or cut, the stone or calculus.

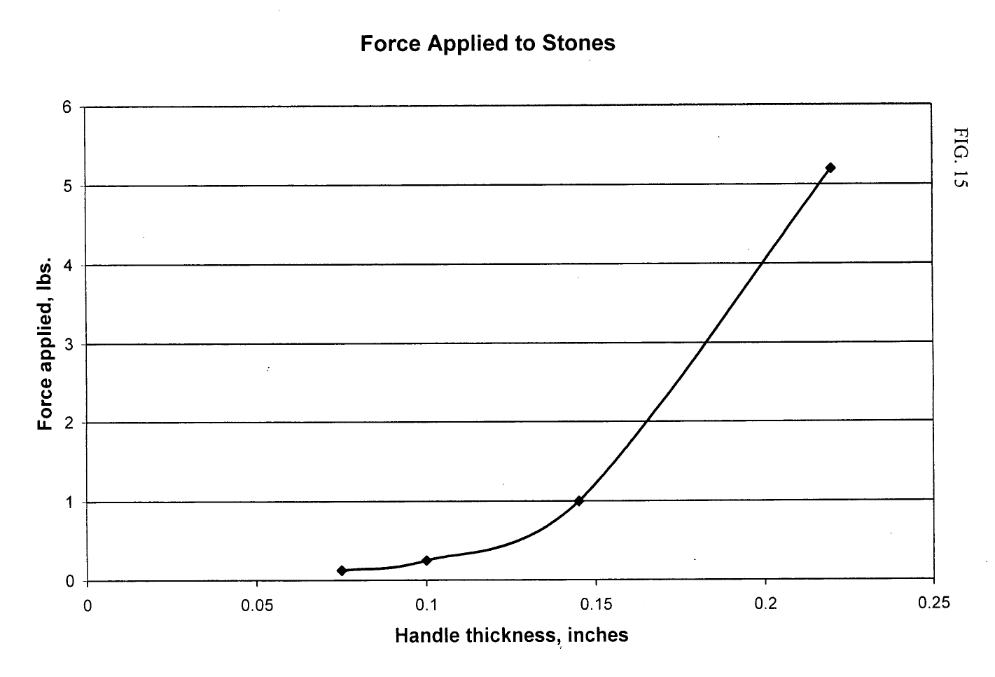

A series of experiments was conducted with a rigid extractor similar to the embodiment of FIG. 1 to determine the force available at the basket with a series of plastic handles similar to those in FIG. 1. The material used was Nylon 6, and the thickness of the handle was varied from about 0.075 inches to about 0.28 inches. The basket was hooked to a mechanical load cell to measure the force, and the result of the experiments is shown in FIG. 15. The data suggest that the force correlates almost linearly with the handle thickness, particularly if the handle thickness is from about 0.15 to about 0.27 inches thick. When the thickness is less than 0.15 inches, the force drops below one or two pounds, and the effects of even small amounts of friction may govern. The force is less predictable in that range.

If the handle is more than about 0.25 to 0.27 inches thick, it may require a force in excess of eight or ten pounds to extend the basket, making the handle and the extractor difficult to operate. It is also clear, that besides varying the handle thickness, the material may be varied, with materials of a lower flexural modulus of elasticity requiring less force while material having a higher flexural modulus will require more force. The shape of the handle cross-section may also be varied, such as by adding ribs or other reinforcing members for a greater force, or by making cuts for a lesser force. Thus, the extractor provides a way to control the force used to extend the basket, and thus also control the force applied to the stone or calculus to be removed. In embodiments using a scissors or jaw-type or grasper-type assembly, the design and selection of the handle allows a user to tailor the cutting or grasping force applied to the object to be removed from a body.

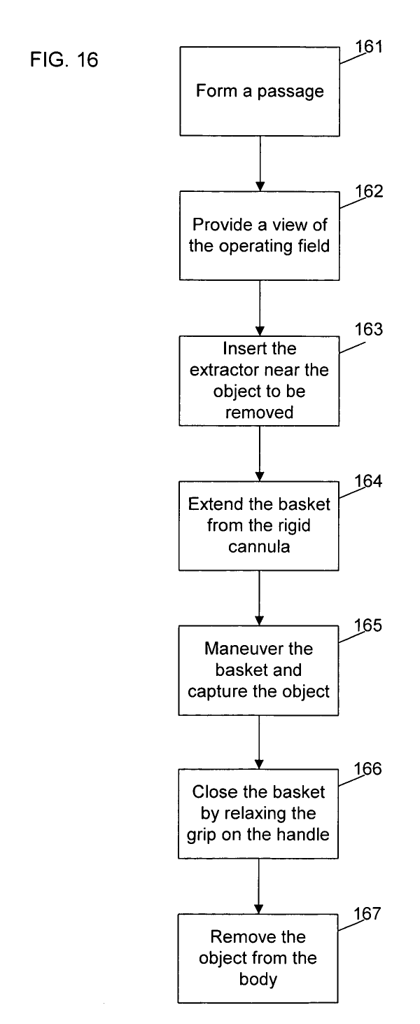

FIG. 16 depicts a method used to remove stones or calculi from a body using the rigid extractor with a tipless, atraumatic basket and a rigid outer cannula. The method comprises a step 161 of forming a passage in order to insert the extractor. The next step 162 is to provide a view of the operating field for the surgeon. As discussed above, the view is preferably provided by a fluoroscope or a nephroscope. The physician then inserts the extractor 163 near the object to be removed, and then extends the basket from the rigid cannula 164. Because the cannula is rigid, it may be moved as desired, even deflecting a nephroscope if one is being used. After the basket is extended, it is necessary to maneuver the basket by using the handle to capture the object 165. The basket is closed by relaxing the grip on the handle 166. Then the object is removed from the body 167.

The embodiments described above are only a few of the ways the invention may be practiced. For instance, the descriptions above have used a fixed outer cannula and a handle with a movable inner cannula attached to the basket, to move the basket forward and out of the outer cannula for deployment. Other variations may be used in which a handle moves the outer cannula backward, with a fixed inner cannula deploying the basket as the outer cannula moves rearward. The surgeon then uses the basket to capture a stone. Such variations may be used in conjunction with a plastic sheath inserted between the outer cannula and the patient. Alternatively, a plastic sheath may also be used with the embodiment of FIG. 1.

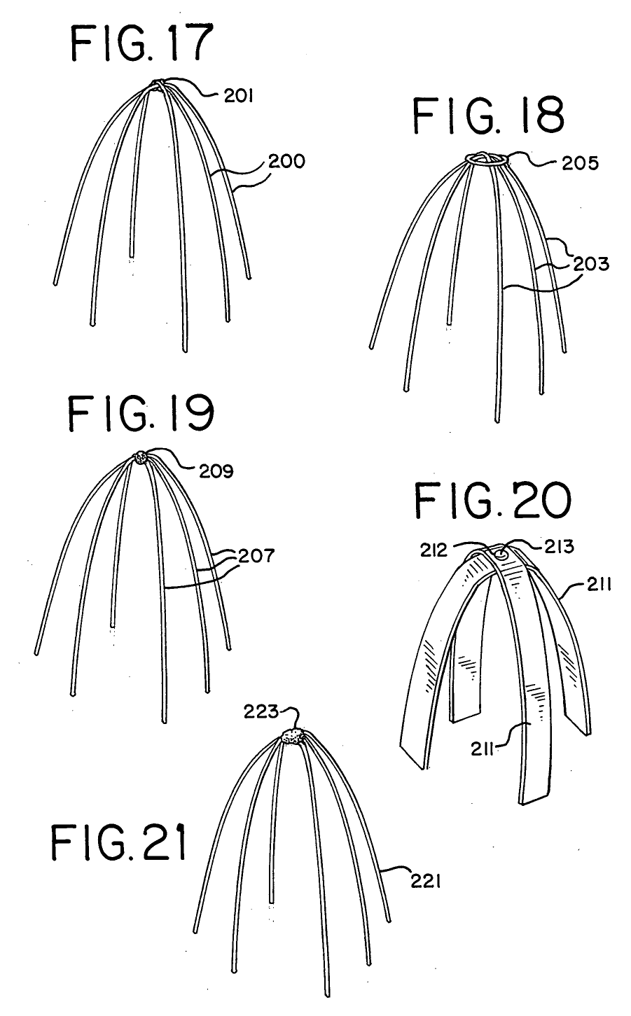

In other embodiments, the basket used for retrieval need not be limited to the tipless basket formed by looping the wires with small loops, as described above. FIGS. 17-21 depict alternate embodiments of baskets useful with the rigid cannula. In FIG. 17 for instance, the wires 200 may be secured by a knot or knots 201 in the wires themselves. In FIG. 18, a separate wire or filament 205 may be used to secure the wires 203 to form a distal end of the basket. Wire, such as Nitinol wire or other medically acceptable wire, such as stainless steel, may be used. Filaments, such as those made from suture material, or other medically-acceptable material, may also be used.

Other techniques may also be used, as shown in FIG. 19, to join wires 207 by using solder joints, braze joints, or weld joints, thus joining the wires to form a distal end of the basket. As shown in FIG. 20, it is even possible to drill holes 212 and use a rivet 213 to join the distal ends of wires 211, to form a distal end of the basket. The rivet embodiment is better accomplished with flat wire than with round wire. Other embodiments, as shown in FIG. 21, may use a small elastomeric or plastic fastener or ball 223 to join the distal ends of wires 221 to form a distal end of a basket useful in the rigid extractor embodiments of the present invention.

Accordingly, it is the intention of the applicants to protect all variations and modifications within the valid scope of the present invention. It is intended that the invention be defined by the following claims, including all equivalents. Since the foregoing detailed description has described only a few of the many alternative forms this invention can take, it is intended that only the following claims, including all equivalents, be regarded as a definition of this invention.

What is claimed is:

1. An extractor for removing an object from a location within a body, the extractor comprising:
an inner cannula;
at least one superelastic wire leg attached to the inner cannula;
at least one superelastic wire loop attached to the inner cannula; and
means for restricting relative movement of the at least one wire loop and the at least one wire leg, the movement restricting means being continuously and kinklessly formed with at least one of the at least one wire leg and the at least one wire loop, wherein the at least one superelastic wire leg, the at least one superelastic wire loop, and the means for restricting relative movement form a reversibly collapsible, tipless, atraumatic basket; and
a rigid outer cannula for delivering the basket to the object.

2. The extractor of claim 1, wherein the means for restricting movement comprises a first smaller loop formed in a wire selected from the group consisting of the at least one wire loop and the at least one wire leg.

3. The extractor of claim 1, wherein the means for restricting movement comprises a first smaller loop formed in a wire loop and a second smaller loop formed in a second wire selected from the group consisting of the at least one wire loop and the at least one wire leg.

4. The extractor of claim 1, wherein the means for restricting movement is formed inside the basket.

5. The extractor of claim 1, wherein the basket further comprises at least a second superelastic wire loop incorporating the at least one wire leg.

6. The extractor of claim 5, wherein the movement restricting means further comprises a second smaller loop formed in the second wire loop.

7. The extractor of claim 1, wherein the basket includes a Nitinol alloy.

8. The extractor of claim 1, further comprising a handle having a first end and a second end, the first end attached to the outer cannula and the second end attached to the inner cannula.

9. The extractor of claim 1, further comprising a handle having a first end and a second end, the first end fixedly attached to the outer cannula and the second end removably attached to the inner cannula.

10. The extractor of claim 8, wherein the basket is collapsed within the outer cannula when the handle is in a relaxed state and wherein the basket is extended from the outer cannula by applying from about 2 pounds of force to about 8 pounds of force to the handle.

11. The extractor of claim 8, wherein the handle is made from plastic and is from about 0.15 inches to about 0.30 inches thick and is from about 0.125 inches to about 0.75 inches wide.

12. The extractor of claim 1, wherein the at least one superelastic wire leg and the at least one superelastic wire loop are in a relaxed condition when the basket is extended from the outer cannula.

13. The extractor of claim 1, wherein the at least one superelastic wire leg and the at least one superelastic wire loop are secured to the inner cannula with at least one of a crimp and an adhesive.

14. The extractor of claim 1, wherein the outer cannula is sufficiently rigid to deflect a nephroscope when used to retrieve and extract a kidney stone.

15. The extractor of claim 1, wherein an outer diameter of the outer cannula is from about 0.110 to about 0.200 inches (about 0.27 to about 0.51 cm), and a wall thickness of the outer cannula is from about 0.010 inches to about 0.020 inches (about 0.025 cm to about 0.051 cm).

16. The extractor of claim 1, wherein the basket is from about 0.8 to about 1.25 inches long (about 2 to 3 cm) and from about 0.4 to about 0.8 inches wide (about 1 to 2 cm).

17. The extractor of claim 1, wherein the outer cannula further comprises an outer plastic covering.

18. The extractor of claim 1, further comprising an outer plastic access sheath for inserting the extractor.

19. A method for removing an object from within a body, the method comprising:
forming a passage with at least one of a needle, a wire guide, and an access sheath;
inserting a rigid extractor near the object;
extending a basket from the rigid extractor and maneuvering the basket near the object;
capturing the object with the basket; and
removing the object from the body.

20. The method of claim 19, further comprising inserting a nephroscope within the passage.

21. The method of claim 20, further comprising deflecting the nephroscope with the rigid extractor to maneuver the basket near the object.

22. The method of claim 19, further comprising controlling a force used to capture the object.

23. A rigid extractor for removing an object from a location within a body, the extractor comprising:
an inner cannula;
at least one superelastic wire leg attached to the inner cannula;
at least one superelastic wire loop attached to the inner cannula;
at least one smaller loop formed in a wire selected from the group consisting of the at least one superelastic wire leg and the at least one superelastic wire loop, the at least one smaller loop restricting relative movement of the at least one superelastic wire leg and the at least one superelastic wire loop, wherein the at least one superelastic wire leg, the at least one superelastic wire loop, and the at least one smaller loop form a reversibly collapsible, tipless, atraumatic basket;
a rigid outer cannula for delivering the basket to the object, the rigid outer cannula having a plastic outer sheath, and the rigid outer cannula containing the inner cannula; and
a handle having a first end and a second end, the first end attached to the outer cannula and the second end attached to the inner cannula.

24. The extractor of claim 23, wherein the handle is made from nylon and is from about 0.15 to about 0.30 inches thick and from about 0.125 inches to about 0.75 inches wide.

25. The extractor of claim 23, wherein the at least one superelastic wire leg and the at least one superelastic wire loop are secured to the inner cannula with at least one of a crimp and an adhesive.

26. The extractor of claim 23, wherein the basket includes a Nitinol alloy.

27. The extractor of claim 23, wherein the basket is collapsed within the outer cannula when the handle is in a relaxed state and wherein the basket is extended from the outer cannula by applying from about 2 pounds of force to about 8 pounds of force to the handle.

28. The extractor of claim 23, wherein the outer cannula is sufficiently rigid to deflect a nephroscope when used to retrieve and extract a kidney stone.

29. The extractor of claim 23, further comprising an outer plastic access sheath for inserting the extractor.

30. The extractor of claim 23, wherein the basket is from about 0.8 to about 1.25 inches long (about 2 to 3 cm) and from about 0.4 to about 0.8 inches wide (about 1 to 2 cm).

31. The extractor of claim 23, wherein the outer cannula further comprises an outer plastic covering.

32. A rigid extractor for removing an object from a location within a body, the extractor comprising:
an inner cannula;
a retrieval assembly attached to the inner cannula;
a rigid outer cannula for delivering the retrieval assembly to the object, the rigid outer cannula containing the inner cannula; and
a handle having a first end and a second end, the first end attached to the outer cannula and the second end attached to the inner cannula.

33. The extractor of claim 32, wherein the handle is made from nylon and is from about 0.15 to about 0.30 inches thick and from about 0.125 inches to about 0.75 inches wide.

34. The extractor of claim 32, wherein the retrieval assembly includes a Nitinol alloy.

35. The extractor of claim 32, wherein the retrieval assembly is stored within the outer cannula when the handle is in a relaxed state and wherein the retrieval assembly is extended from the outer cannula by applying from about 2 pounds of force to about 8 pounds of force to the handle.

36. The extractor of claim 32, wherein the outer cannula is sufficiently rigid to deflect a nephroscope when used to retrieve and extract a kidney stone.

37. The extractor of claim 32, further comprising an outer plastic access sheath for inserting the extractor.

38. The extractor of claim 32, wherein the outer cannula further comprises an outer plastic covering.

39. The extractor of claim 32, wherein the retrieval assembly is selected from the group consisting of a basket, a pair of jaws, and a pair of scissors.

40. A rigid extractor for removing an object from a location within a body, the extractor comprising:
an inner cannula;
a basket made of wires attached to the inner cannula;
a rigid outer cannula for delivering the basket to the object, the rigid outer cannula containing the inner cannula; and
a handle having a first end and a second end, the first end attached to the outer cannula and the second end attached to the inner cannula.

41. The extractor of claim 40, wherein the handle is made from nylon and is from about 0.15 to about 0.30 inches thick and from about 0.125 inches to about 0.75 inches wide.

42. The extractor of claim 40, wherein the basket includes a Nitinol alloy.

43. The extractor of claim 40, wherein the basket is stored within the outer cannula when the handle is in a relaxed state and wherein the basket is extended from the outer cannula by applying from about 2 pounds of force to about 8 pounds of force to the handle.

44. The extractor of claim 40, wherein the outer cannula is sufficiently rigid to deflect a nephroscope when used to retrieve and extract a kidney stone.

45. The extractor of claim 40, further comprising an outer plastic access sheath for inserting the extractor.

46. The extractor of claim 40, wherein the outer cannula further comprises an outer plastic covering.

47. The extractor of claim 40, wherein the handle is made from plastic and is from about 0.15 to about 0.30 inches thick.

48. The extractor of claim 40, wherein the basket further comprises at least one connector connecting the wires at a distal end of the basket, the connector selected from the group consisting of a loop, a knot, a braze joint, a solder joint, a weld joint, a rivet, a filament, a wire, an elastomeric ball, and a plastic ball.

49. The extractor of claim 40, wherein the wires of the basket are in a relaxed condition when the basket is extended from the outer cannula.

50. A method for removing an object from a body, the method comprising:
forming a passage with at least one of a needle, a wire guide, and an access sheath;
inserting a rigid extractor near the object;
extending a retrieval assembly from the rigid extractor and maneuvering the retrieval assembly near the object;
capturing the object with the retrieval assembly; and
removing the object from the body.

51. The method of claim 50, further comprising inserting a nephroscope within the passage.

52. The method of claim 50, further comprising deflecting the nephroscope with the rigid extractor to maneuver the retrieval assembly near the object.

53. The method of claim 50, further comprising controlling a force used to capture the object.
